(19) United States
(12) Patent Application Publication
(10) Pub. No.: US 2026/0215924 A1
(43) Pub. Date: Jul. 30, 2026

(54) IMPLANTABLE VENTILATION SYSTEM

(71) Applicant: BREATHIN LTD., Haifa (IL)

(72) Inventors: Elias HELLOU, Haifa (IL); Shahar PELED, Beit HaShitta (IL); Gideon MEIRY, Kibbutz Shomrat (IL); Omri NAVEH, Alon Hagalil (IL)

(21) Appl. No.: 19/145,484

(22) PCT Filed: Jan. 14, 2024
(86) PCT No.: PCT/IL2024/050051

§ 371 Date: Jul. 2, 2025

Related U.S. Application Data
(60) Provisional application No. 63/439,138, filed on Jan. 15, 2023.

(51) Int. Cl. A61F 2/30; A61F 2/48

(52) U.S. Cl. CPC A61F 2/30749; A61F 2/482; A61F 2/484

(57) ABSTRACT

Provided herein are implantable mechanical ventilation systems and methods for improving, supporting and/or treating ventilation insufficiencies in a subject in need thereof.

### TECHNICAL FIELD

The present invention relates to implantable mechanical systems and methods for improving, supporting and/or treating ventilation insufficiencies in subjects in need thereof.

### BACKGROUND

Mechanical ventilation by maneuvering the chest wall is an old paradigm, dating to the 1930s when the first automatic ventilator, the iron lung, was developed to support ventilating patients who suffered from Poliomyelitis. As the chest wall expands, it decreases the pressure inside the pleural space, forcing air inspiration via the ventilation airways followed by relaxation upon the chest wall returning to its resting position, increasing the pressure within the pleural space which pushes out air from the lungs (expiration).

This spontaneous brain-controlled consequence of chest wall maneuver and diaphragm contraction followed by relaxation is the normal periodic sequence of spontaneous ventilation. Thus, mechanical ventilation can theoretically be executed either by forcing movement of the chest wall or maneuvering the diaphragm, as well as by inflating and deflating the lungs as modern mechanical ventilators do.

Mechanical maneuvering of the chest is a valuable manipulation under certain conditions, including, for example: Patients suffering from neurodegenerative conditions can sometimes still breathe on their own but are incapable of full chest wall expansion; A chest wall mechanical maneuvering device can be effective in weaning from mechanical ventilation, for example, by taking just a partial effort of the breathing effort, such a device may help to trigger self-spontaneous breathing.

The mechanism underlying patients becoming used to a mechanical ventilator and needing weaning is not fully cleared. It may involve breathing muscles weakening during long mechanical ventilation and/or effects of long mechanical ventilation on the breathing control in the brain, which may impair its control of spontaneous breathing pacing. Studies suggest that non-invasive ventilation after early extubation helps in reducing the total days spent on invasive mechanical ventilation, and that the less time spent on invasive ventilation had lower rates of ventilator-associated-pneumonia. In some cases, weaning is rapid and uneventful, however, for some patients the process may be prolonged for days or weeks.

Moreover, some patients require mechanical ventilation, either partially or completely due to a medical condition, and/or the progression of a medical condition. However, confining a patient to bed and attaching them to a positive pressure ventilation may reduce the patient's quality of life, e.g., by limiting mobility, limiting their ability to communicate, causing conditions associated with prolonged confinement to bed, further weakening of the patient's muscular skeletal system, as well as increasing the financial burden on the patient and/or the medical institution.

Therefore, there is a need for cost effective, efficient, and relatively easy to implement implantable mechanical system for supporting and/or treating ventilation insufficiencies.

### SUMMARY

According to some embodiments, provided herein are implantable systems and methods for improving or supporting breathing, and/or for treating various ventilation insufficiencies in subjects in need thereof.

According to some embodiments, the implantable systems disclosed herein are advantageous as they are cost effective to produce and operate, safe to use, portable (by means of being implantable thus, facilitating portability of the subject); and highly efficient in improving, supporting and/or treating ventilation insufficiencies, at various settings, including, for example, for weaning from non-portable ventilation systems, or other breathing supportive therapies, for improving self-breathing in patients, for improving subject's ability to talk, for improving subject ability to cough, for aiding breathing in subjects afflicted with a neurodegenerative condition, and the like.

According to some embodiments, and without wishing to be bound to any theory or mechanism, the implantable mechanical ventilation system disclosed herein can affect the volume of the thoracic cavity of the subject by inducing physical/mechanical relative movement of bones of the thoracic cage, wherein the controlled changes in the volume of the thoracic cavity can induce lung ventilation, by forming negative pressure and decreasing the pleural pressure leading to airflow through the patient airways into the lungs.

According to some embodiments, the implantable mechanical ventilation system disclosed herein generally includes a moveable unit, which is configured to be attached/fixed/anchored to anterior bone(s) (such as sternum) of the thoracic cavity of the subject on the one hand and be associated (physically and/or functionally) with an actuator, which is anchored/held in position by a static unit (inside or outside of the rib cage). Accordingly, when the actuator is activated (for example, under the regulation of an external controller), it is configured to induce a relative movement (displacement) of the moveable unit, which in turn induces movement of the associated bone(s), thereby changing the size of the rib cage and the volume of the thoracic cavity. The volume change of the thoracic cavity in turn, thereby affects lung volume/activity, by facilitating at least partial inhalation or exhalation, respective to the volume change of the thoracic cavity. According to some embodiments, the system may further include one or more biosensors, that can aid in providing biological signals for activating movement of the moveable unit, such that the breathing is coordinated based on physiological signals.

According to some embodiments, there is provided herein a mechanical system for improving, supporting and/or treating ventilation insufficiencies in a subject in need thereof, the system includes: a moveable unit configured to be attached/fixed to an anterior bony structure of a thoracic cavity, said moveable unit is physically and/or functionally associated with an actuator; and a static component configured to anchor the actuator, and be connected to a posterior section of at least one rib; wherein, when activated, the actuator is configured to displace the moveable unit and the attached bony structure, thereby affecting the volume of the thoracic cavity.

According to some embodiments, affecting the volume of the thoracic cavity may include increasing the cavity volume, thereby forming negative pressure inducing inhalation into the lungs and/or decreasing the volume cavity, thereby inducing exhalation from the lung.

According to some embodiments, the actuator may include a stationary motor; and a moveable arm or sliding elements at least partially positioned on top of each other. According to some embodiments, the arm or sliding elements are located posteriorly or inferiorly to the moveable unit.

According to some embodiments, the actuator and the static component may be positioned subcutaneously, externally to rib cage of the subject.

According to some embodiments, the actuator and the static component may be positioned within rib cage of the subject.

According to some embodiments, the motor may include an electric motor, pneumatic piston or a hydraulic piston.

According to some embodiments, the system may further include a diaphragm harness configured to prevent suction of the diaphragm into the thoracic cavity.

According to some embodiments, the system may further include a controller configured to activate the actuator.

According to some embodiments, the system may further include one or more biosensor(s) configured to detect initiation of spontaneous or ventilator-induced inspiration of the subject.

According to some embodiments, the one or more biosensor(s) may be selected from: an oxygen sensor, a carbon dioxide sensor, a pressure sensor, a stretch sensor, an accelerometer, an electrical sensor to detect nerve and/or muscle stimulation or activity, electromyography sensor (EMG), or any combination thereof. Each possibility is a separate embodiment.

According to some embodiments, the one or more biosensors may be positioned externally and/or internally in the subject's body.

According to some embodiments, the system may further include a malfunction sensor, selected from, an accelerometer, a voltmeter, a current sensor, or any combinations thereof.

According to some embodiments, the moveable unit may include a fixation plate rigidly fixed to the anterior bony structure.

According to some embodiments, the moveable unit may be composed of a biocompatible material.

According to some embodiments, the biocompatible material may be selected from, but not limited to: stainless steel, cobalt chrome alloys, titanium and titanium alloy, pyrolytic carbon, thermoplastics, bioceramics, or any combination thereof. Each possibility is a separate embodiment.

According to some embodiments, the static component may include a strap. In some embodiments, the strap may be rigid, semi-rigid or flexible.

According to some embodiments, the at least one strap may include two portions, each of the portions is configured to be anchored posteriorly at one end to at least one rib and anchored anteriorly at the other end to the actuator.

According to some embodiments, the at least one strap may be anchored posteriorly at one end to at least one rib by a plate and/or a hinge.

According to some embodiments, the bony structure is an anterior rib or sternum of the subject.

According to some embodiments, the moveable unit may include a fixation plate configured to be attached to or fixed onto the bony structure of the thoracic cavity, wherein the relative vertical movement of the moveable unit, induced by the actuator, induces volume change of the thoracic cavity.

According to some embodiments, at least part of the system is configured to be implanted subcutaneously.

According to some embodiments, the controller may be configured to activate the actuator at a predetermined range of motion, predetermined timing and/or at a predetermined rate. Each possibility is a separate embodiment.

According to some embodiments, the controller may be configured to activate the actuator at a range of motion, timing and/or rate, based on information from one or more sensors. Each possibility is a separate embodiment.

According to some embodiments, the controller may be configured to activate the actuator to expand the volume of the thoracic cavity by a predetermined amount, at predetermined timing and/or at a predetermined rate. Each possibility is a separate embodiment.

According to some embodiments, the controller may be configured to activate the actuator to expand the volume of the thoracic cavity, by an amount, timing and/or rate determined based on information from one or more sensors. Each possibility is a separate embodiment.

According to some embodiments, the controller may be configured to be worn externally by a user.

According to some embodiments, the system may further include a power source, such as, rechargeable battery.

According to some embodiments, the rechargeable battery may be configured to be worn externally by a user.

According to some embodiments, the system is portable.

According to some embodiments, there is provided a method for supporting, improving and/or treating ventilation insufficiencies in a subject in need thereof, the method includes:
implanting the ventilation system as disclosed herein in the subject; and
using the controller to control operating parameters of the ventilation system.

According to some embodiments, operating parameters may include range of motion, expansion volume of the thoracic cavity, timing of activation, rate of activation, timing of expansion of the thoracic cavity, rate of expansion of the thoracic cavity, or any combinations thereof. Each possibility is a separate embodiment.

According to some embodiments, the method may be used for supporting weaning of a subject from breathing support devices.

According to some embodiments, the method may be used for improving self-breathing of the subject.

According to some embodiments, the method may be used for facilitating talking and/or coughing of the subject.

According to some embodiments, the subject may be afflicted with a neurodegenerative condition.

According to some embodiments, there is provided a surgical method for implanting a system as disclosed herein, the surgical method includes the steps of:
fixing the moveable unit onto the anterior bony anterior bony structure of the thoracic cavity of the subject;
positioning the static component and the actuator;
associating the moveable unit and the actuator; and
controlling the operation of the actuator by an external controller, such that when activated, the actuator is configured to displace the moveable unit, thereby inducing movement of the bony structure and affecting the volume of the thoracic cavity;
to thereby at least partially control breathing of the subject.

Certain embodiments of the present disclosure may include some, all, or none of the above advantages. One or more other technical advantages may be readily apparent to those skilled in the art from the figures, descriptions, and claims included herein.

Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A shows a schematic illustration of an implantable mechanical ventilation system, according to some embodiments;

FIG. 1B shows a schematic illustration of the implantable mechanical ventilation system of FIG. 1A, when positioned on a rib cage, and connected to a controller, according to some embodiments;

FIG. 2A shows a schematic illustration of an implantable mechanical ventilation system, according to some embodiments;

FIG. 2B shows a schematic illustration of the implantable mechanical ventilation system of FIG. 2A, when positioned on a rib cage, according to some embodiments;

FIG. 3A shows a schematic illustration of a perspective view of an implantable mechanical ventilation system, in a resting (closed) position, according to some embodiments;

FIG. 3B shows a side view cross section of the implantable mechanical ventilation system, in a resting position, according to some embodiments;

FIG. 3C shows a schematic illustration of a perspective view of an implantable mechanical ventilation system, in an open (pulled) position, according to some embodiments;

FIG. 3D shows a side view cross section of the implantable mechanical ventilation system, in an open (pulled) position, according to some embodiments;

FIG. 4A shows a schematic illustration of a perspective close-up view of an implantable mechanical ventilation system, in a resting (retracted position), according to some embodiments;

FIG. 4B shows a schematic illustration of a front view of an implantable mechanical ventilation system, when positioned on a rib cage, in a resting (retracted position), according to some embodiments;

FIG. 4C shows a schematic illustration of a perspective close-up view of an implantable mechanical ventilation system, in an open, according to some embodiments;

FIG. 4D shows a schematic illustration of a front view of an implantable mechanical ventilation system, when positioned on a rib cage, in an open, according to some embodiments;

FIG. 5 shows a schematic illustration of an implantable mechanical ventilation system, at least partially positioned inside the rib cage, according to some embodiments;

FIG. 6A shows a schematic illustration of an implantable mechanical ventilation system, according to some embodiments;

FIG. 6B shows a schematic illustration of the implantable mechanical ventilation system of FIG. 6A, at least partially positioned inside the rib cage, according to some embodiments;

FIG. 6C shows a schematic illustration of a perspective view of the implantable mechanical ventilation system, in a resting (closed) position, according to some embodiments;

FIG. 6D shows a schematic illustration of a perspective view of an implantable mechanical ventilation system, in an open (extracted) position, according to some embodiments;

FIG. 6E shows a schematic illustration of a perspective view of an actuator of an implantable mechanical ventilation system in a resting (closed position), according to some embodiments;

FIG. 6F shows a schematic illustration of a perspective view of an actuator of an implantable mechanical ventilation system in an open (extracted position), according to some embodiments;

FIG. 7A shows a schematic illustration of components of an implantable mechanical ventilation system, with respect of a rib cage, according to some embodiments;

FIG. 7B shows a schematic illustration of an implantable mechanical ventilation system, in a resting position compared to an open position, according to some embodiments;

FIG. 7C shows a schematic illustration of a perspective view of components of an implantable mechanical ventilation system with respect of rib structure, according to some embodiments;

FIG. 7D shows a schematic illustration of a perspective view of an implantable mechanical ventilation system, at least partially positioned in the rib cage, according to some embodiments;

FIG. 7E shows a schematic illustration of a perspective view of an implantable mechanical ventilation system, positioned on the rib cage, according to some embodiments;

FIG. 8 shows a schematic illustration of rib connection means, according to some embodiments;

FIG. 9A shows a schematic illustration of perspective view of a diaphragm harness, according to some embodiments;

FIG. 9B shows a schematic illustration of perspective view of a diaphragm harness, positioned in a rib cage, according to some embodiments;

FIG. 10A shows a schematic illustration of exemplary biosensors position with respect of a ventilation system, according to some embodiments;

FIG. 10B shows a schematic illustration of perspective view of exemplary biosensors position with respect of a chest cavity of a subject, according to some embodiments; and

FIG. 11 shows a flow chart of steps in breath regulation/control using an implantable mechanical ventilation system, according to some embodiments.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

In the following description, numerous details are set forth for the purpose of explanation. However, one of ordinary skill in the art will realize that the invention may be practiced without the use of these specific details.

As used herein, the term “ventilation” may relate to the process of exchange of air between the lungs and the ambient air.

As used herein, the term “weaning” relates to the gradual withdrawal of a patient from assisted breathing on a life-support system or other form of mechanical respiratory treatment and/or therapy.

As used herein, the terms “standalone ventilator” relates to a ventilation device/system that can supply adequate Tidal Volume and Minute Ventilation gas exchange to support patient metabolic needs, without requiring additional equipment.

As used herein, the term “supportive ventilator” relates to a ventilator that does not completely rely on as the device that supplies the entire gas exchange of a patient is a ventilation-supportive device. Some supportive ventilators may enrich the inhaled air with oxygen whether on spontaneous or another device-induced ventilation, and/or may assist in mechanical motion of a patient's lungs, chest, diaphragm, etc.

As used herein, the term “positive pressure ventilator” relates to a ventilation device which inflates the lungs with air, typically through a ventilation tube that is either intubated or inserted by tracheostomy.

As used herein, the term “negative pressure ventilation” relates to a ventilation device/system that typically surrounds externally the patient's chest and periodically applies negative pressure which pulls the chest wall, expanding it and by doing so, decreases the pleural pressure leading to airflow through the patient airways into their lungs (similarly to the mechanism of spontaneous breathing). For example, the Iron Lung was the first developed negative pressure ventilator. A subsequent negative pressure ventilator is, for example, the HAYEK ventilator.

As used herein, the term “Tidal Volume” (“TV”) relates to the volume of air inhaled.

As used herein, the term “Minute Ventilation” (“MV”) relates to the volume of air that is exchanged during one minute, that is, the Tidal Volume X number of ventilation cycles per minute.

As used herein, the term “posterior” is directed to the back side of the body.

As used herein, the term “anterior” is directed to the front side of a body (for example.

As used herein, the term “superior” is directed to a location/position that is closer to the head of a body (for example, a body of a subject). In some embodiments, the term superior is interchangeable with “proximal”.

As used herein, the term “inferior” is directed to a location which is farther from the head of a body (or is closer to the lower part of the body). In some embodiments, the term inferior is interchangeable with “distal”.

As used herein, the terms “top” and “bottom” refer to top and bottom directions of a subject body. In some embodiments, “top” is relatively closer to the head region and “lower” is relatively closer to the leg region. In some embodiments, top and bottom are relative to the transverse plane (axial or horizontal plane), which divides the body into cranial and caudal (head and tail) portions.

As used herein, the terms “subject” and “patient” may interchangeably be used.

According to some embodiments, the implantable mechanical ventilation system disclosed herein may be used as a standalone ventilator and/or a ventilation supportive device. According to some embodiments, the mechanical ventilation system may be used to provide temporary, continuous and/or permanent ventilation.

According to some embodiments, the ventilation system disclosed herein, may be used for weaning a subject from supportive ventilation treatment.

According to some embodiments, as further detailed below herein, the ventilation system may facilitate pulling the sternum of the subject (to which it is attached), in a vector which may represent normal sternum movement during spontaneous breathing. According to some embodiments, pulling the sternum periodically, and then allowing it to return to its resting position, may mimic spontaneous breathing cycles. According to some embodiments, pulling the sternum may expand the chest, thereby generating negative pressure in the pleural space, which may lead to the inspiration of air into the lungs through the upper airways. According to some embodiments, the Tidal Volume of air inhaled during expansion of the sternum may depend on the distance to which the sternum is expanded.

Advantageously, the ventilation system disclosed herein may be configured to provide full and/or partial ventilation assistance.

Advantageously, in accordance with some embodiments, in full control of ventilation configuration, the ventilation system disclosed herein may fully replace the mechanical work/action of the breathing muscles. According to some embodiments, the ventilation system may solely maneuver the patient's chest to produce adequate Tidal Volume and Minute Ventilation. According to some embodiments, full control of ventilation may be required when a patient's own spontaneous breathing mechanism may not be functioning, and the patient may entirely depend on a ventilator to fully control gas exchange and/or breathing.

Advantageously, in some embodiments, in partial control of ventilation configuration, the mechanical ventilation system may be operated to assist a patient's own spontaneous ventilation efforts and/or may assist another ventilator, which may also be connected to the patient and may ventilate the patient.

According to some embodiments, in partial control of ventilation configuration, the ventilation system may assist a patient who breathes spontaneously, but whose spontaneous breathing does not produce adequate Tidal Volume and Minute Ventilation. According to some embodiments, in partial control of ventilation configuration, the ventilation system may aid the patient's own breathing efforts, e.g., by an extra chest wall dilatation, which may bring in an extra amount of air during inhalation. According to some embodiments, in partial control of ventilation configuration, the ventilation system may begin to pull the chest wall at any time during the spontaneous inhalation, e.g., at the beginning of the spontaneous inspiration, and/or at any point during the inhalation. According to some embodiments, in partial control of ventilation configuration, the ventilation system may ignore the self-spontaneous force generated by the patient's own breathing muscles and may apply full expansion force (e.g., pulling).

According to some embodiments, the ventilation system may include several units that, when operating in synchronization, may support the action of inhalation and/or exhalation.

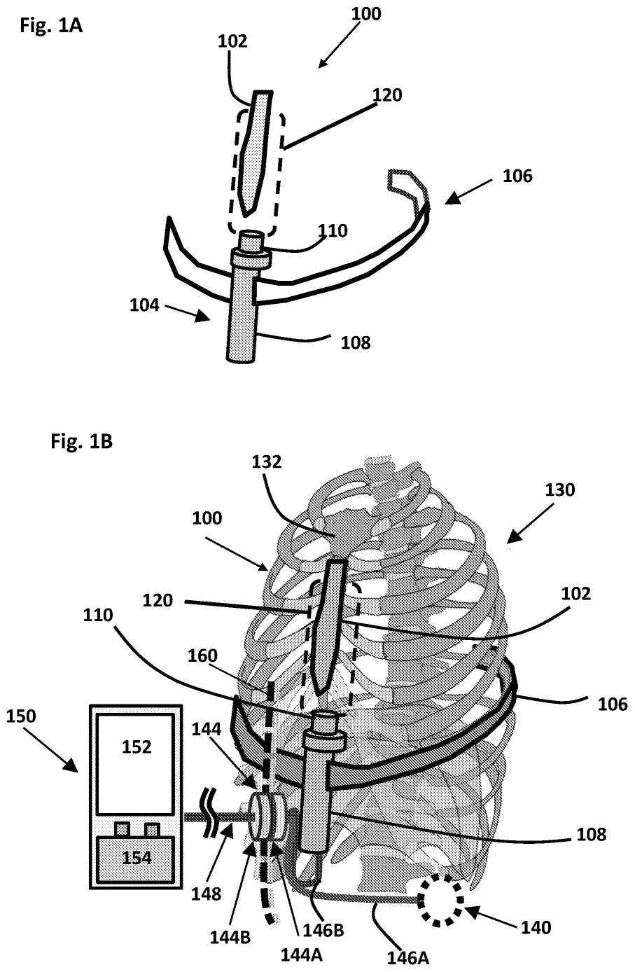

Reference is now made to FIG. 1A, which shows a schematic illustration of an implantable mechanical ventilation system, according to some embodiments. As shown in FIG. 1A, system 100 generally includes a moveable unit (element) 102, which is configured to be attached (permanently or transiently) to an anterior bony structure of the subject (not shown), in particular, to the rib cage. The attachment to the rib cage may be facilitated by any suitable means, including, for example, but not limited to by gluing, using fixation means (such as screws, etc.). In some embodiments, the moveable unit may include a fixation plate which is configured to be fixated to the bony structure and thereby secure the unit to the bone. In some embodiments, as detailed herein below, the bony structure is a sternum, or at least a portion of the sternum. As further detailed below, the moveable unit is configured to move relatively to actuator 104, whereby such movement induces movement of the connected bony structure, and hence, the rib cage volume (i.e., chest cavity volume) may change accordingly. Actuator 104 is associated (physically and/or functionally) with moveable unit 102, such that moveable unit may translate between various positions, as illustrated in FIG. 1A by dashed box 120, illustrating the range of vertical motion of the moveable unit 102. As shown in FIG. 1A, actuator 104 includes at least two parts: gear (also referred to as “arm”) 110 and motor 108. Gear 110 shown in FIG. 1A is configured to move vertically (up-down positions), such that when hitting/touching/engaging moveable element 102, it can facilitate the respective movement of moveable element 102. The movement of gear 110 is facilitated by motor 108, which may be any type of motor (e.g., electrical motor, magnetic motor, hydraulic motor, and the like). The operation of the motor itself is controlled by a controller (not shown), as further detailed below. Further shown in FIG. 1A is static unit 106 of system 100. Static unit 106 is configured to anchor/hold/place actuator 104 to at least one rib (not shown). In some embodiments, the at least one rib to which the actuator is placed, is not a chest cage rib. As detailed herein, actuator 104 is anchored anteriorly (distally) to the moveable element and the rib cage. Static element 106 may include any type of suitable anchoring element, including, for example, a belt or a strap, that may latch on, or be attached to a rib, to thereby secure the static element to place. As further detailed herein below, the static element may include a plurality or combination of elements, including, for example a plurality of belts, straps, latches, hinges, hooks, and the like.

Reference is now made to FIG. 1B which shows a schematic illustration of the implantable ventilation system of FIG. 1A, when positioned on a rib cage, and connected to a controller, according to some embodiments. As shown in FIG. 1B, system 100 is placed on an internal body portion of a subject. In particular, moveable unit 102 is fixed to sternum 132 of rib 130. Static element 106, which is used to anchor/place actuator 104, is associated posteriorly with a rib, at both ends thereof, and at an anterior region, with actuator 104. Further shown in FIG. 1B, is external controller (control unit) 150. External controller 150 is configured to control operating parameters of the system, including, for example, the timing, rate and/or degree of movement of the actuator, thereby controlling the timing, rate and/or degree of volume change of the chest cavity. As detailed below, the controller may include one or more processors, a memory, a display, a user interface (shown as exemplary user interface 152), a power source (shown as exemplary battery 154), and the like. The controller unit may communicate directly or indirectly, wirelessly or using wires with one or more units of the system. For example, as shown in FIG. 1B, controller unit 150 may communicate with one or more internal sensors, such as exemplary sensor 140. Such sensors may provide information/data regarding the breathing of the subject and/or regarding operation of the various units of the system, and such data may be used by the controller unit to determine/adjust corresponding operating parameters. In the example shown in FIG. 1B, the sensor may communicate/convey information with the controller unit via communication unit 144. Communication unit 144 may include any type of communication unit configured to transfer data, information, power between various units. In some embodiments, communication unit 144 may include at least two subunits: a first subunit 144A, which is placed internally, within the subject body, and a second subunit 144B, which is placed externally to the subject body (Dashed line 160 schematically illustrates the border line between external and internal parts of the body). The two subunits can communicate wirelessly or using wires (for example, via penetrating wire), such that information, data or other signals (such as, electrical power) from inside-outside the body can be easily, efficiently and safely conveyed. In the setting illustrated in FIG. 1B, data from the sensor 140 is conveyed to internal subunit 144A of communication unit 144, via wires (shown as communication wires 146A), and that data is conveyed wirelessly to external subunit 144B, which in turn can convey the information (wirelessly, or using wires), to controller unit 150. Likewise, the communication unit 144 can be used to convey information to/from the controller to any unit of the system, for example, to actuator 104. As shown, actuator 104 may be connected by wires (shown as power and communication wires 146B) to internal subunit 144A of communication unit 144. In such a setting, controller unit can control operation of actuator 104, via communication unit 144, by providing operating instructions (e.g., timing of operation of the actuator (i.e., when will the gear move), rate of operation (at what frequency will the gear move), and extent of operation (to what extent/distance will the gear move). In some embodiments, the controller may provide operating parameters which are predetermined (for example, according to a predetermined treatment plan, that may be general or adjusted to the subject). In some embodiments, the controller may provide operating parameters based on real time information/data received and processed. In some embodiments, a user (for example, a health care provider) may determine one or more operating parameters, based, for example, on condition and characteristics of the subject, type of plan, type of treatment, and the like. In some embodiments, the user may communicate remotely with controller unit (for example, by Wi-Fi connection, Bluetooth confection, and the like).

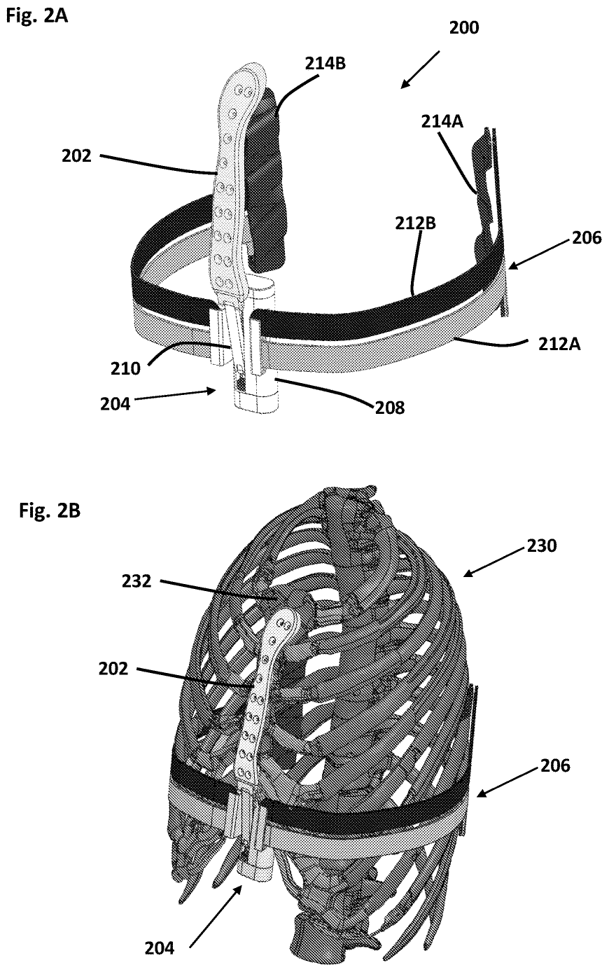

Reference is now made to FIG. 2A, which shows a schematic illustration of an implantable mechanical ventilation system, according to some embodiments. As shown in FIG. 2A, system 200, generally includes a moveable unit (element) 202, which is configured to be attached (permanently or transiently) to an anterior bony structure of the subject (not shown), in particular, to the rib cage. The attachment to the rib cage may be facilitated by any suitable means, including, for example, but not limited to by gluing, using fixation means (such as screws, etc.). In some embodiments, the moveable unit may include a fixation plate which is configured to be fixated to the bony structure and thereby secure the unit to the bone. In some embodiments, as detailed herein below, the bony structure is a sternum, or at least a portion of the sternum. As detailed herein, the moveable unit is configured to move relatively to actuator 204, whereby such movement induces movement of the connected bony structure, and hence, the rib cage volume (i.e., chest cavity volume) may change accordingly. As shown in FIG. 2A, actuator 204 includes at least two parts: gear (also referred to as “arm”) 210 and motor 208. Gear 210 shown in FIG. 2A is connected to a distal region of moveable unit 202, such that when gear 210 rotationally moves about an axis, it facilitates the respective movement of moveable unit 202, such that moveable unit 202 can extend anteriorly, thereby by “pulling” and extending the rib cage and hence increasing the thoracic cage volume. Likewise, when the gear returns to its initial (resting) position, the moveable unit also returns to a resting position, thereby reducing (i.e. returning/restoring) the volume of the thoracic cavity. The movement of gear 210 is facilitated by motor 208, which may be any type of motor (e.g., electrical motor, magnetic motor, hydraulic motor, and the like). The operation of the motor itself is controlled by a controller (not shown), as further detailed herein. Further shown in FIG. 2A is static unit 206 of system 200. Static unit 206 is configured to anchor/hold/place actuator 204 to at least one rib (not shown). Static element 206 may include any type of suitable anchoring element, including, for example, a belt or a strap, that may latch on, or be attached to a rib, to thereby secure the static element to place. As shown in FIG. 2A, static element 206 includes at least two straps, an upper (superior strap) 212B and a lower (inferior) strap 212A, wherein each of the straps may be continuous, or may be composed of two parts, each part extending from a posterior rib to the actuator 204. In addition, anchoring elements 214A-B may also be used as part of the static unit, to allow anchoring/attaching/securing the static unit to posterior ribs, for example, by being placed between ribs.

Reference is now made to FIG. 2B, which shows a schematic illustration of the implantable mechanical ventilation system of FIG. 2A, when positioned on a rib cage, according to some embodiments. As shown in FIG. 2B, system 200 is placed on an internal body portion of a subject. In particular, moveable unit 202 is fixed, at a proximal region thereof, to sternum 232 of rib 230. Static element 206 (including straps 212A-B) is associated posteriorly with a rib, at both ends thereof, and at an anterior region, with actuator 204. Also shown are anchoring elements 214A-B of static unit 206, each is associated with posterior ribs regions. Not shown in FIG. 2B, is external controller (control unit), which is functionally and/or structurally similar to controller unit 150 described in FIG. 1B.

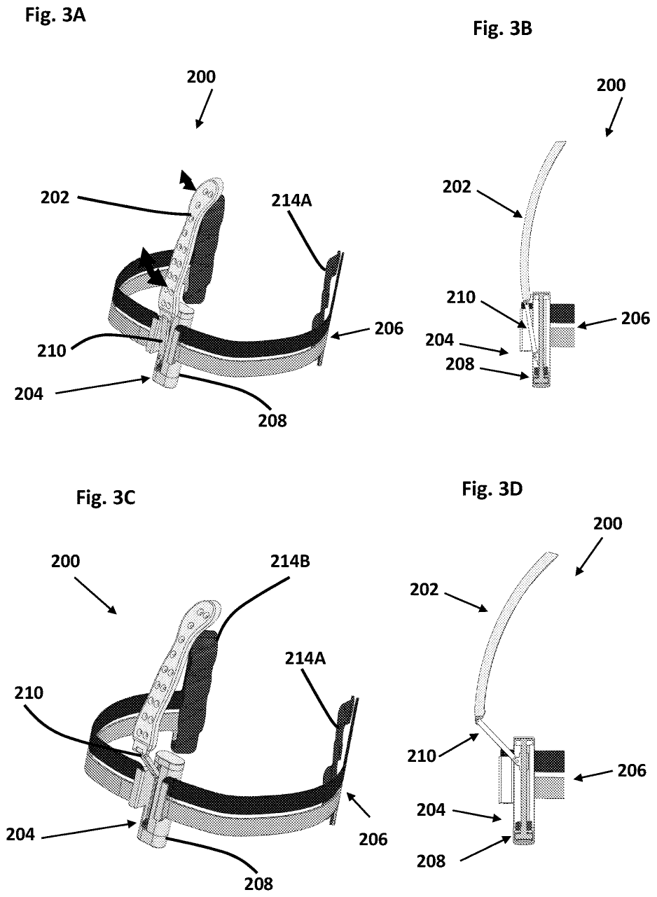

Reference is now made to FIGS. 3A-D, which show a schematic illustration of a perspective and cross section side-view, respectively, of the implantable mechanical ventilation system of FIG. 2A, in a resting (closed) position (FIGS. 3A-B) and at an open (expended) position (FIGS. 3C-D), according to some embodiments. As shown in FIGS. 3A-D, moveable unit 202 is configured to move away/towards the rib cage at the distal portion thereof (i.e., the region connected to the actuator), by virtue of the pulling/pushing/releasing movement of the actuator 204 (in particular, arm 210). As illustrated in the cross-section side view presented in FIG. 3B, at the resting (closed) position, arm 210 is positioned at a closed/folded position within its housing such that moveable element 202 is in a resting position as well, whereby the rib cage bones are not pulled by the moveable unit. As shown in FIGS. 3C-D, when the arm 210 of actuator 204 is moved upwards and outwards, this facilitates the movement of moveable unit 202, such that the distal region thereof (which is connected to the actuator) moves/pushed in a frontal direction, forcing movement of the proximal region thereof, which is fixed to the rib cage (not shown). Thus, by the movement of arm 210, a relative movement of the associated moveable unit 202 is induced, which in turn would lead to a change in volume of the rib cage. In other words, upon pulling, or otherwise affecting the position of the moveable unit, the rib cage volume is affected.

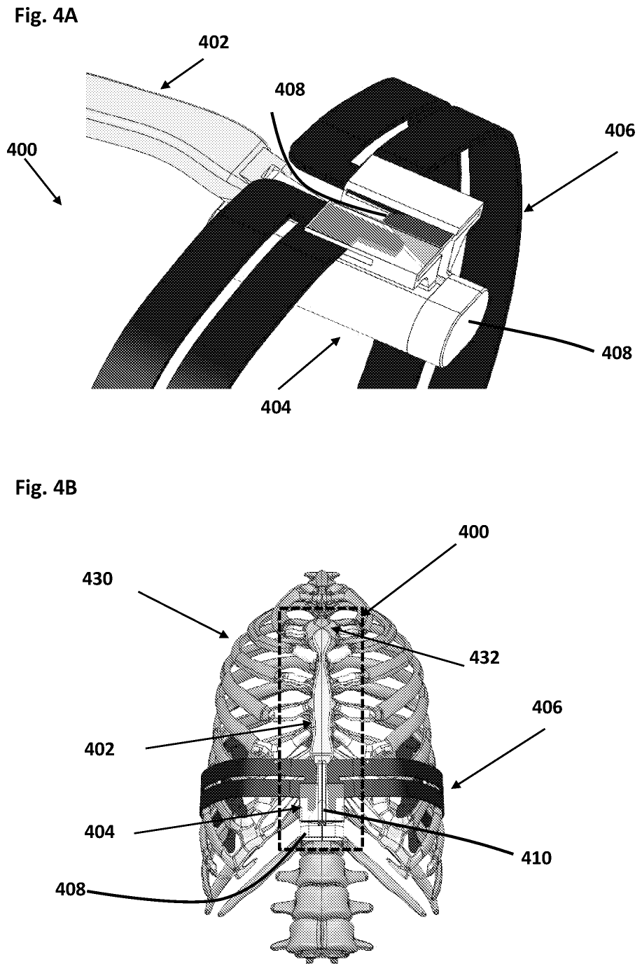

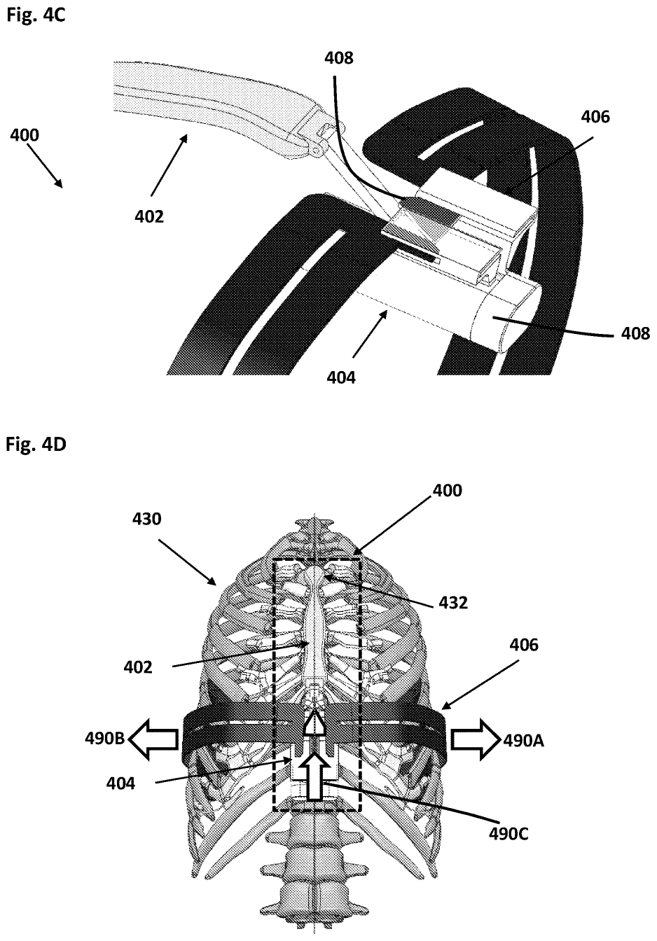

Reference is now made to FIGS. 4A-D, which show schematic illustrations of an implantable mechanical ventilation system in a closed position (FIGS. 4A-B) and in an open position (FIGS. 4C-D), according to some embodiments. As shown in FIGS. 4A and 4C, system 400, generally includes a moveable unit (element) 402, which is configured to be attached (permanently or transiently) to an anterior bony structure of the subject (not shown), in particular, to the sternum of the rib cage. As detailed herein, the moveable unit is configured to move relatively to actuator 404, whereby such movement induces movement of the connected bony structure, and hence, the rib cage volume (i.e., chest cavity volume) may change accordingly. As shown in FIG. 4A, actuator 404 includes at least two parts: motor 408 and gear (also referred to as “arm” or “pin”) 410. Gear 410 shown in FIG. 4A is in the form of a pin, and is positioned at a “closed” (retracted”) position. The pin may be connected to a distal region of moveable unit 402. The actuator is positioned using static component 406. Shown in FIG. 4C is mechanical ventilation system 400, in an open position, e.g. when pin 410 of actuator 420 is pushed superiorly, is facilitates the respective superior movement (displacement) of moveable unit 402, thereby extending the rib cage and hence increasing the thoracic cage volume. FIG. 4B illustrates system 400 in a closed position, when placed on rib cage 430, whereby moveable unit 402 is anchored to sternum 432. FIG. 4D illustrates system 400 in an open position, when placed on rib cage 430.

When the arm (pin) 410 of actuator 404 is moved upwards and, this facilitates the displacement of moveable unit 402, such that the distal region thereof (which is connected to the actuator) moves/pushed in a frontal direction, forcing movement of the proximal region thereof, which is fixed to the rib cage. By the relative movement of moveable unit 202, rib cage 430 is expanded, as illustrated by arrows 490A-C, thereby increasing the volume thereof. As shown, the static unit may be configured to expand as well, to accommodate the expansion of the rib cage. In other words, upon pulling, or otherwise affecting the position of the moveable unit, the rib cage volume is affected, for example, by inducing rib expansion due to force applied by the moveable unit on the sternum.

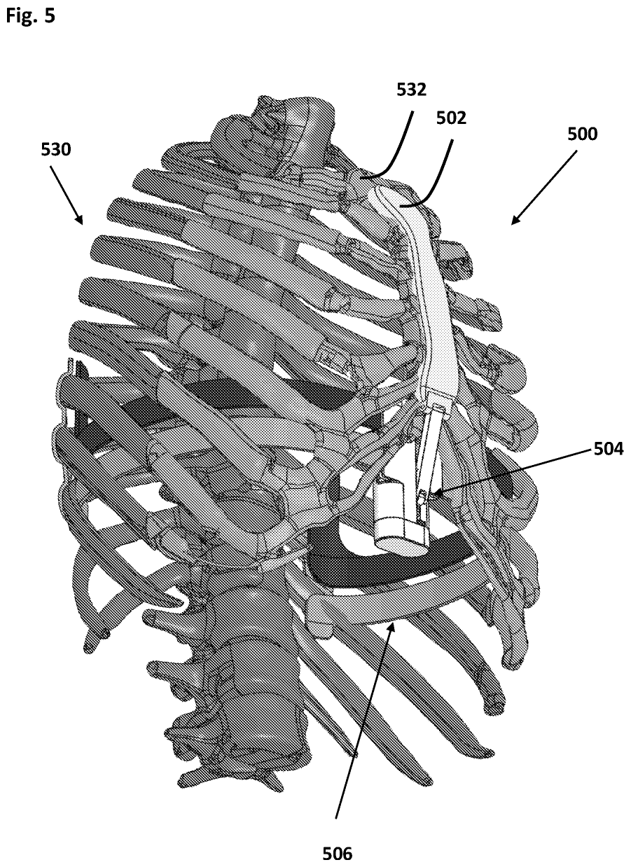

Reference is now made to FIG. 5, which shows a schematic illustration of an implantable mechanical ventilation system, at least partially positioned inside the rib cage, according to some embodiments. As shown in FIG. 5A, mechanical ventilation system 500 may be at least partially positioned within the rib cage 530. In particular, static unit 506 and actuator 504 may be positioned internally to the rib cage, such that static unit 506 is at least partially engaging inner surface of at least some ribs of rib cage 530. Moveable unit 502 of system 500 is positioned/anchored/fixed externally to the rib cage, on a bony structure thereof, such as, the sternum 532. Thus, while the actuator and the static unit are positioned/anchored within the internal portion of the rib cage, the moveable unit is positioned externally, and the displacement thereof, induced by movement of the gear of the actuator induced rib cage enlargement, and hence enhancement of the thoracic cavity volume. As further detailed hereinbelow, the static unit may be anchored (internally) to posterior ribs by an anchoring element configured to fit in-between adjacent ribs.

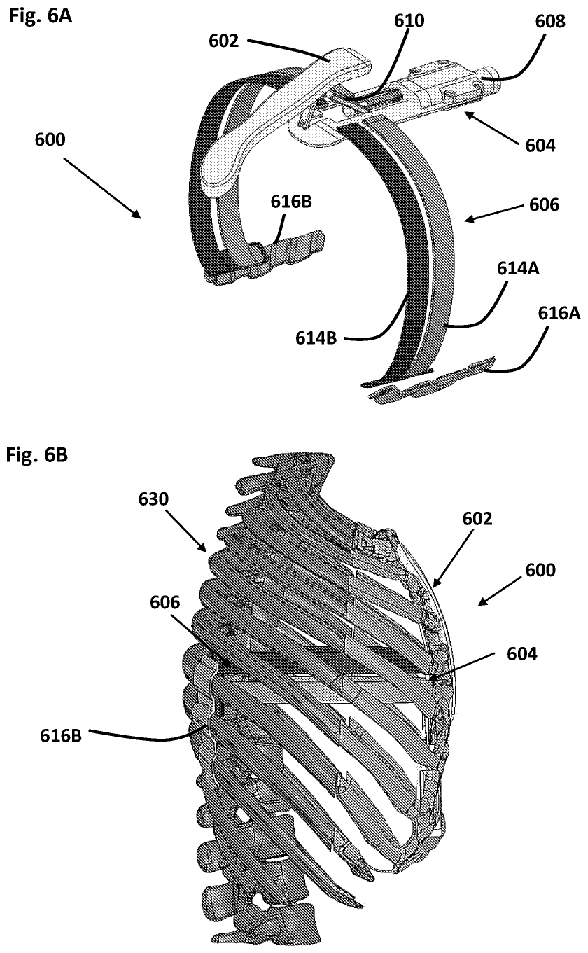

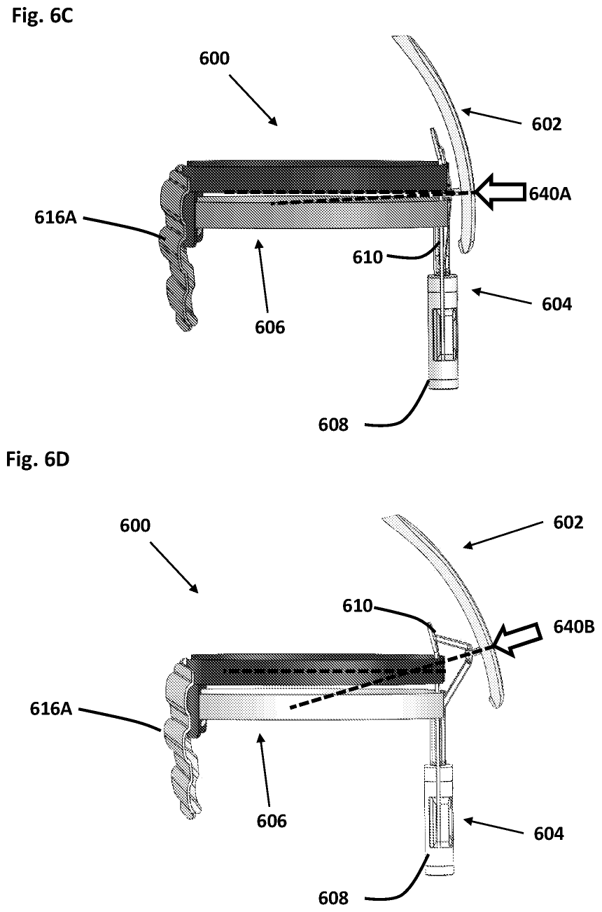

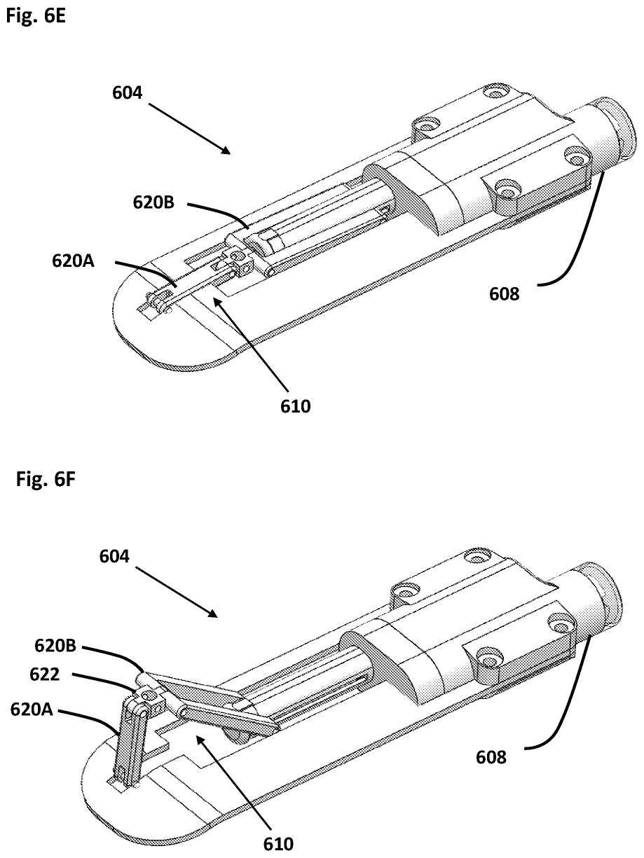

Reference is now made to FIG. 6A, which shows a schematic illustration of an implantable mechanical ventilation system, according to some embodiments. As shown in FIG. 6A, system 600 includes a moveable unit 602, actuator 604; having at least a gear 610 and motor 608. The gear is configured to interact with moveable unit 602 (which is configured to be anchored to a bony structure (e.g. sternum), and to induce movement/displacement thereof, for example, by pushing the unit (from anterior towards posterior positions). Further shown is static unit 606, which includes straps or belts 614A-B, and posterior anchoring elements 616A-B, which are each configured to anchor/hold the static component (unit) 606 to ribs of the subject. Shown in FIG. 6B system 600, whereby static unit 606 and actuator 604 are positioned within the rib cage (i.e., facing internal surface of the ribs), while moveable unit 602 is positioned on the external surface of the rib cage, for example, on the sternum.

Reference is now made to FIGS. 6C-6D which show schematic illustrations of side views of an implantable mechanical ventilation system, in a resting (closed) position and in an open (pushed) position, according to some embodiments. As shown in FIGS. 6C-6D, while actuator 604 and static unit 606 do not change their relative position, regardless of the position of moveable unit, by virtue of the movement of arm 610 of actuator 604, displacement of moveable unit 602 is facilitated, which in turn would affect the relative size/volume of the thoracic cavity. In particular, as shown in FIG. 6C, when in closed (retracted position), the moveable unit 602 assumes a natural position, with respect of the rib cage (as marked by dashed lines and arrow 640A). As shown, moveable unit 602 is connected to arm 610 of actuator 604 (which is located within the rib cage). In the open position illustrated in FIG. 6D, arm 610 is in an open position, thereby displacing moveable unit 602. The displacement induces posterior-superior shift of the moveable unit (and hence the rib cage, by pulling the sternum), as indicated by the dashed lines and arrow 640B. In some embodiments, the sternum may be displaced at an angle of 90°, relative to the back of the subject. Thus, by reciprocal movement of arm 610 (activated by motor 608 and controlled by, for example, a controller), between closed and open positions (the timing and/or degree of which may be controllable), the volume of the thoracic cavity may be changes/controlled.

Reference is now made to FIGS. 6E-6F which show a schematic illustration of a perspective view of the actuator of an implantable mechanical ventilation system in a resting (closed) position and in an open position, respectively. As shown in FIGS. 6E-F, actuator 604 includes at least a motor 608, and moveable arm 610. Moveable arm may be moveable between a closed/retracted/pulled position, as shown in FIG. 6E. When activated by motor 608 (and controlled by a controller, as detailed herein), arm 610 may be pushed, such that the arm can extends upwards/forward (e.g., to protrude out of the rib cage (where the connected moveable unit is located)), thereby pushing the connected moveable unit, as illustrated in FIGS. 6C-D. As shown in FIGS. 6E-F, arm 610 may be composed of several portions (for example, portions 620A-B), that are hinged thereto (for example, at region 622), thereby facilitating the extending of the arm at a desired extent and direction.

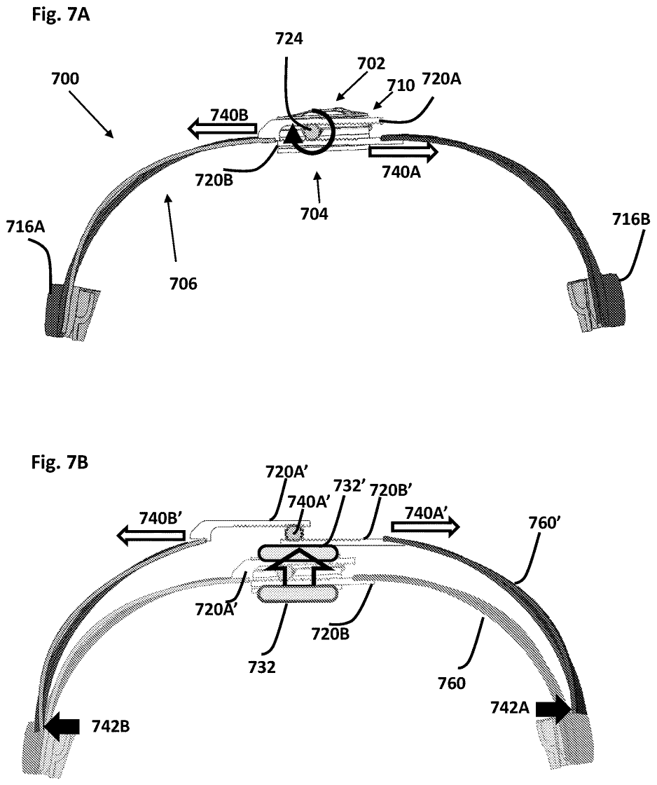

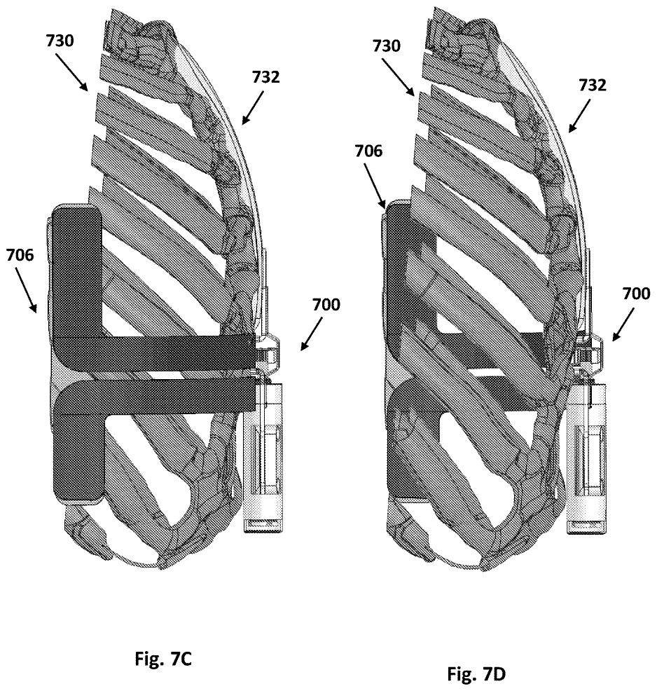

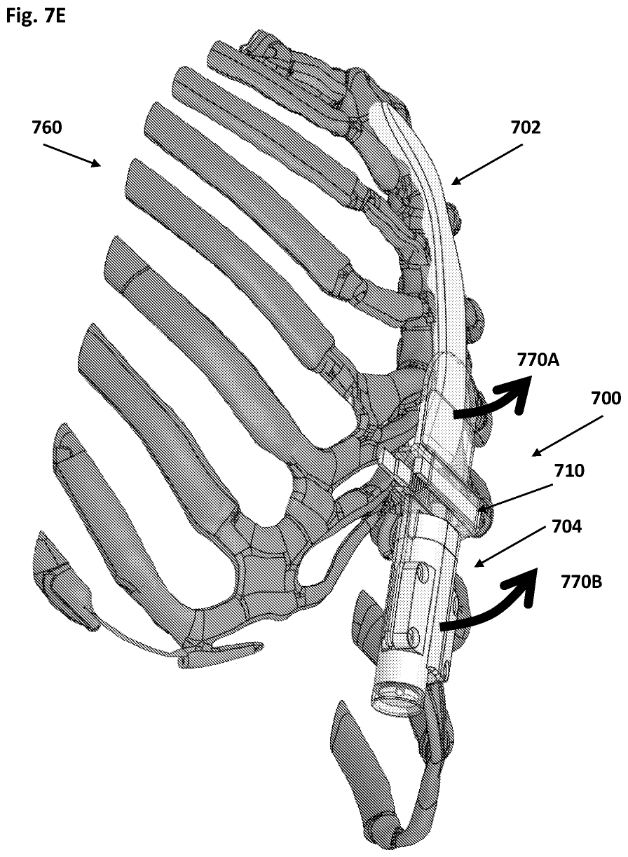

Reference is now made to FIG. 7A, which shows a schematic illustration of components of an implantable mechanical ventilation system, with respect of a rib cage, according to some embodiments. As shown in FIG. 7A, system 700 generally includes a moveable unit (element) 702, which is configured to be attached (permanently or transiently) to an anterior bony structure of the subject (not shown), in particular, to the rib cage. In some embodiments, the moveable unit may include a fixation plate which is configured to be fixated to the bony structure and thereby secure the unit to the bone. In some embodiments, as detailed herein below, the bony structure is a sternum, or at least a portion of the sternum. As shown in FIG. 7A, the actuator 704 includes at least two parts: movable plates or platforms) 720A-B (also collectively referred to herein as arm 710) and motor 708. Arm 710 shown in FIG. 7A is composed of two separate movable plates/platforms 720A-B, located one on top of the other (i.e., a first plate 720A is positioned superiorly or anteriorly relative to second plate 720B). The plates, which may be substantially parallel thereto, are configured to move along a horizontal axis (side-ways), such that when the plates are moving in opposite directions (i.e. in the respective direction of arrows 740A-B), elevation of the arm, induces corresponding movement of moveable unit 702, connected thereto, (i.e. move anteriorly and superiorly), thereby moving the connected sternum and inducing enlargement of the thoracic cavity, as further detailed herein below. In such a setting, the moveable means is elevated due to the side-extension of the ribs, induced by the actuator. The relative movement of the plates may be facilitated by any suitable means, including, for example, a ratchet mechanism, operable by a gear wheel 724. The movement of the gear wheel is controlled by motor 708 of actuator 704, and the operation of the motor itself is controlled by a controller (not shown), as detailed herein. As shown in FIG. 7A, when the gear wheel is rotated in the direction of the circular arrow, the plates move in opposing directions, and when the gear wheel rotates counter-wise, the plates move towards each other. Further shown in FIG. 7A is static unit (components) 706 of system 700. Static unit 706 is configured to anchor/hold/place actuator 704 to at least one rib (not shown). Static element 706 may include any type of suitable anchoring element, including, for example, a belt or a strap, that may latch on, or be attached to a rib, to thereby secure the static element to place. As further detailed herein below, the static element may include a plurality or combination of elements, including, for example a plurality of belts, straps, latches, hinges, hooks, and the like. In the example shown in FIG. 7A, static unit 706 is made of at least two portions of belts, and anchoring elements 716A-B, at the posterior ends thereof, configured to anchor the straps to the ribs.

Reference is made to FIG. 7B which shows a schematic illustration of implantable mechanical ventilation system 700, in resting position (760) compared to open position (760′), wherein the two illustrated positions are superimposed (overlaid), to illustrate the change in relative position of the moveable unit and the rib cage. As shown in FIG. 7B, when plates 720A-B (closed position) are moved in opposite directions (marked by arrows 740A′-740B′), to assume open positions 720A′-B′, the moveable element is moved, along with sternum 732, that moves anteriorly-superiorly (to assume position 732′), inducing the lateral movement of ribs, such that collectively, rib cage 760 (resting position) is enlarged to assume open position 760′. In the configuration illustrated in FIGS. 7A-B, the actuator may move together with the moveable unit and the sternum. FIG. 7C shows a schematic illustration of a perspective view of components of system 700 with respect of rib structure 750. As shown in FIG. 7C, when activated, arm 710 of actuator 704 induces anterior-superior movement (in the direction of arrows 770A-B) of moveable unit 702 along with the connected sternum. As shown in FIG. 7C, in such configuration, both the actuator and the moveable unit are configured to be displaced together, along with the sternum. According to some embodiments, system 700 may be positioned externally to the rib cage 730, as illustrated in FIG. 7C, or internally, within the rib cage 730 (at least with respect of static unit 706), as illustrated in FIG. 7D.

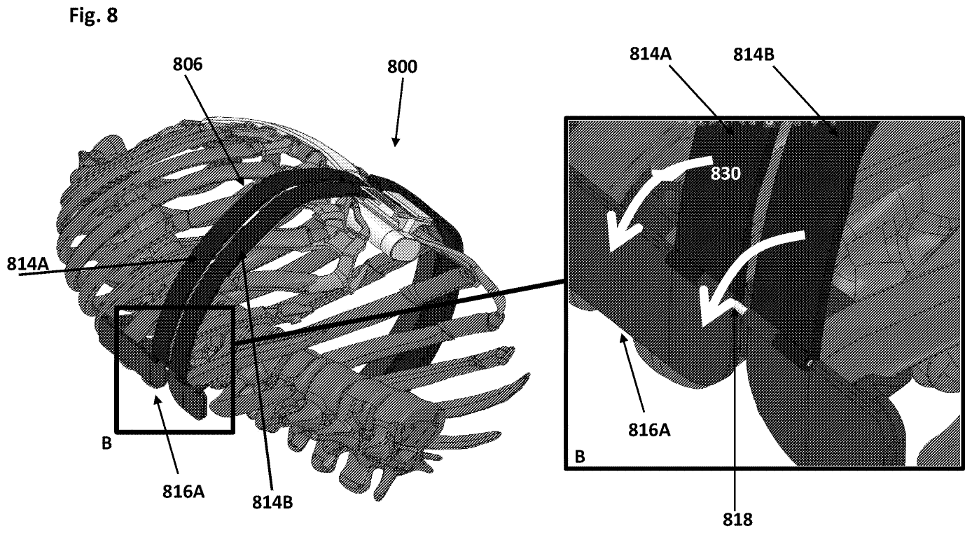

According to some embodiments, the static components (unit) may include anchoring means, for anchoring a posterior portion thereof to one or more ribs. In some embodiments, the anchoring means may include hinges, allowing the pivotal movement of the static unit, to accompany or facilitate the opening movement of the rib cage. Reference is now made to FIG. 8 which shows a schematic illustration of rib connection means, according to some embodiments. As shown in FIG. 8, system 800 is configured to be positioned on the rib cage, such that static components 806 (which may be comprised of any suitable means, as detailed herein), is attached/anchored/associated with posterior portions of the ribs. To this aim, one or more anchoring means may be used. Shown in FIG. 8 is exemplary anchoring means 816A. Anchoring means 816A is configured to be attached to/in between ribs and allow connection to straps 814A-B of static component 806. The connection between anchoring means 816A and straps 814A-B may be rigid or may be flexible. In some embodiments, the connection may be flexible, such that the straps may be at least partially rotatable with respect of the anchoring means. As shown in the enlarged view in panel B, anchoring means may be attached by hinges to straps 814A-B. In such a setting, the straps may at least partially rotate around hinge 820, in the direction marked by white arrows 730, thereby aiding in the opening of the rib cage.

According to some embodiments, the connection/attachment/anchoring to the ribs may be to any suitable rib. For example, the rib may be any one of claims 8-10. In some embodiments, the rib is rib 10. In some embodiments, the attachment/fixation point/region may be a posterior-medial rib. According to some embodiments, the system disclosed herein may facilitate sternum movement of about 10-50 mm. In some embodiments, the system disclosed herein may facilitate sternum movement of about 20-40 mm. In some embodiments, the system disclosed herein may facilitate sternum movement of about 30-35 mm. In some embodiments, such induced movement can result in sufficient volume increase of the thoracic cavity, to at least partially induce breathing of the subject. In some embodiments, a tidal volume of the subject may be in the range of 300-600 ml, or any subrange thereof.

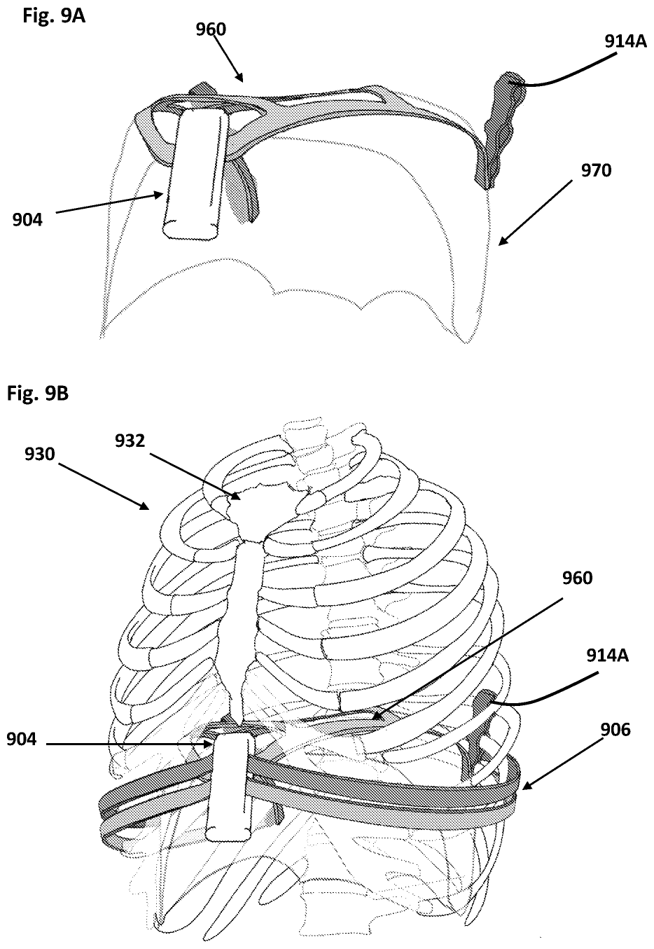

According to some embodiments, the system disclosed herein may further include a diaphragm harness. In some embodiments, the harness may be part of the system. In some embodiments, the diaphragm harness may be used as an optional add-on to the system. In some embodiments, the use of the diaphragm harness is configured to prevent suction of the diaphragm into the thoracic cavity. In some embodiments, the diaphragm may gradually lose its rigidity and may compensate for the volume changes induced by the system. Hence, a diaphragm harness may be used. Reference is now made to FIG. 9A, which shows a schematic illustration of a perspective view of a diaphragm harness, according to some embodiments. As shown in FIG. 9A, diaphragm harness 360 may be placed/positioned over diaphragm 970 of a subject, to thereby prevent undesired movement (in particular, suction) of the diaphragm into the thoracic cavity. As shown in FIG. 9A, the harness may be held/fixed in placed by use of static elements 914A-B), capable of anchoring the harness to at least some of the ribs. Further, actuator 904 of a ventilation system (other parts thereof not shown in this illustration) may also be associated with the diaphragm harness. Reference is now made to FIG. 9B which shows a schematic illustration of a perspective view of the diaphragm harness, positioned in a rib cage, according to some embodiments. As shown in FIG. 9B, harness 960 is positioned within the rib cage 930, over (on top of) the diaphragm, such that it can prevent suction thereof. According to some embodiments, the harness may be hard and may be made of any suitable biocompatible material, including, for example, but not limited to: plastic, fiber glass reinforced plastic, polycarbonate, Polyether ether ketone (Peek), poly-plastic polymer, Titanium and the like, or any combinations thereof. Each possibility is a separate embodiment. In some embodiments, the size, shape and/or composition of the harness may be customized to the patient. In some embodiments, the harness is placed within the thoracic cavity, whereas other units of the system (such as, actuator, moveable element) are placed (subcutaneously) on the external surface of the rib cage.

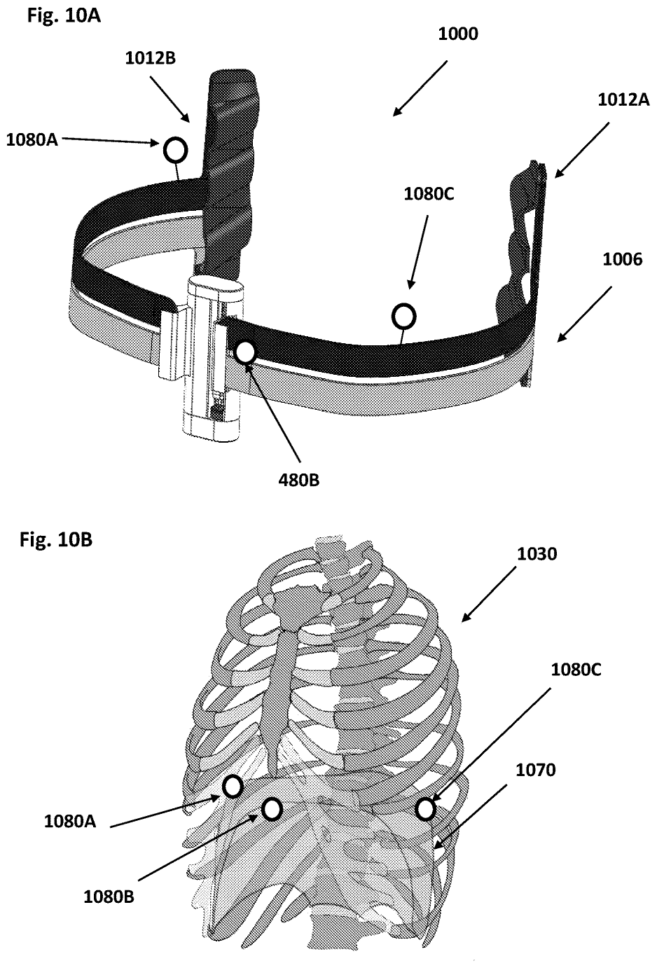

According to some embodiments, as further detailed below herein, the system may further include one or more biosensors that may be used to sense/detect various biological or physiological parameters related to breathing, and based thereon, to allow determining/controlling operation of the ventilation system. According to some embodiments, such sensors may include, for example, but not limited to: an oxygen sensor, a carbon dioxide sensor, a pressure sensor, a stretch sensor, an accelerometer, an electrical sensor to detect nerve and or muscle stimulation or activity (Electromyography sensor (EMG)) or any combination thereof. Each possibility is a separate embodiment. The sensors may be positioned within the units of the system, or at other locations, either internal or external locations on or in association with the subject body. As detailed above, the sensors may communicate with the controller of the system, to convey sensed data, and to allow the controller to determine operating parameters, based at least on that data. Reference is now made to FIGS. 10A-B which shows a schematic illustration of exemplary biosensors position with respect to the ventilation system (FIG. 10A) and the thoracic cavity (FIG. 10B), according to some embodiments. As shown in FIG. 10A, ventilation system 1000 (which may be similar to ventilation system 200 illustrated in FIG. 2A), includes at least a moveable unit 1002, actuator 1004 and static unit 1006. Further shown are sensors 1080A-C, which are associated with the system. Shown in FIG. 10B is the respective physiological position of the sensors, which are placed within close proximity to diaphragm 1070, in rib cage 1030. In the example shown in FIG. 10A, the sensors are physically and functionally associated with the system. For example, the sensor shown in FIGS. 10A-B may include EMG leads (implantable or encapsulated), which are configured to detect breathing muscle movement, and based thereon, to allow the controller to control the timing, extent and/or rate of operation, to facilitate optimal breathing assistance to the subject (for example, based on the condition of the subject, the type of desired treatment (e.g., weaning, supportive), and the like.

According to some embodiments, the system disclosed herein may further include one or more malfunction sensors, configured to detect mal function of various units of the system. In some embodiments, such sensors may include, for example, but not limited to: current meter, accelerometer, voltage meter, thermometer, force meter, stress gauge, and the like, or any combination thereof. Each possibility is a separate embodiment. In some embodiments, each sensor or a plurality of sensors may be associated with various units of the system. For example, a current sensor may be associated with the motor, to detect resistance of the motor (indicative of proper functioning thereof). For example, an accelerometer may be associated with the motor, to detect frequency changes thereof. For example, an accelerometer may be associated with the moveable unit, to detect movement thereof. For example, an accelerometer may be associated with the static unit, to detect movement thereof.

According to some embodiments, the actuator disclosed herein may include a “Shrink and Expand” drive and a connective arm. According to some embodiments, the connective arm may be connected to a moveable unit (fixation plate). According to some embodiments, the moveable unit may be connected to the patient's rib cage, sternum bone and/or spine. According to some embodiments, the moveable unit may be connected to a patient's sternum bone to generate the sternum movements. According to some embodiments, the moveable unit may be similar to a standard sternum surgical fixation plate, may be a subcutaneous magnetic implant attached to the sternum, etc. According to some embodiments, by applying an electrical and/or magnetic field, synchronized to the breathing action of the patient, the pressure on the skin may be controlled e.g., the electrical and/or magnetic field may be applied only during inhalation to eliminate skin ischemia.

According to some embodiments, a dedicated connection between the arm and the moveable unit may not be rigid. According to some embodiments, a dedicated connection between the arm and the moveable unit may be through a hinge and/or joint, which may allow relative movement between the components.

According to some embodiments, the fixation plate and/or fixation frame may connect anteriorly to the sternum. According to some embodiments, posterior connection to the spine may be to the spinal vertebra, e.g., at the pedicle (such as, in spine fixation). According to some embodiments, posterior connection to the spine may be made by two or more artificial ribs (e.g., between one each side to 12 each side, etc.). According to some embodiments, movement by two or more artificial ribs may force the sternum to move upwards to enlarge the rib cage on inhalation.

According to some embodiments, the ventilation system may include a wearable suit. According to some embodiments, the wearable suit may include one or more elastic belts, and/or one or more pads. According to some embodiments, the one or more pads may include a quick lock to enable easy connection and/or operation of the mechanical ventilation system. According to some embodiments, the one or more pads may connect the drive device arms and may distribute the weight and/or force on a patient's body.

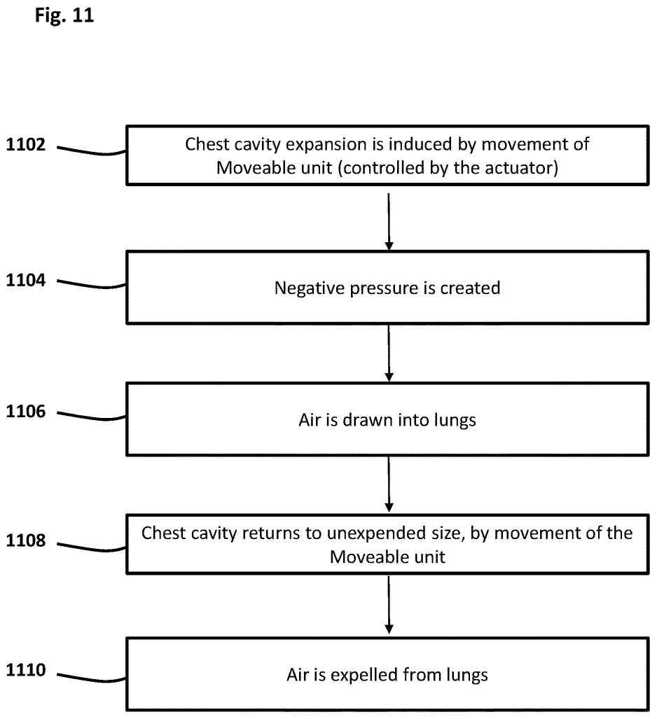

According to some embodiments, as detailed herein, the controller of the system may be used to determine, control and/or affect various operating parameters, to ensure a suitable breathing assistive regime is provided. Reference is now made to FIG. 11, which shows a flow chart of steps in breathing regulation/control using the implantable ventilation system, according to some embodiments. As shown in FIG. 11, at step 1102, a controller of the system provides operating instructions to the actuator, to induce movement of the moveable unit, to thereby cause physical expansion of the chest cavity. The operating instructions may be based on information received from various biosensors (such as, EMG, or others, as detailed above), or autonomously, based on predetermined instructions, (i.e. automatic mode). As a result, at step 1104, negative pressure is formed, which causes, at step 1106, air to be drawn to the lungs. Thereafter, at step 1108, the chest cavity is allowed to return to its unexpended size, by the movement of the moveable unit, which is configured to return to its resting position (or any desired position). As a result of the decrease in the volume of the chest cavity, at step 1110, air is expelled from the lungs. Thus, by each such cycle of steps, a desired breathing cycle is achieved. As detailed above, the degree of activation (i.e., range movement of the moveable unit), the rate of activation (i.e., the number of times per minute the moveable unit is moving) and/or the timing of activation (i.e., when does the moveable unit is moving) are determined by the controller. In some embodiments, the determination of operating parameters by the controller may be based, at least partially, on sensed data. In some embodiments, the determination of operating parameters by the controller may be performed in real-time. In some embodiments, the determination of operating parameters by the controller may be predetermined. In some embodiments, the determination of operating parameters by the controller may be patient specific. In some embodiments, the determination of operating parameters may be adjusted by a user, which may communicate with the controller remotely or physically (for example, via a user interface thereof).

According to some embodiments, the actuator may include a controllable “neutral mode”/“neutral position”, in which the arm is not activated, and is passive, thereby facilitating the option of disengaging the system (for example, to allow natural, uncontrolled breathing by the subject). In some embodiments, the neutral position is used as a safety means. In some embodiments, the neutral position may be facilitated mechanically or physically (for example, by disengaging the association between the motor and the gear, using a clutch, and the like). In some embodiments, the control over the neutral mode may be facilitated by the controller of the system.

In some embodiments, one or more components of the system may be disengaged from the system. In some embodiments, one or more units/components of the system may be removed from the subject. In some embodiments, the actuator and/or the moveable unit may be removed, once treatment has been completed.

According to some embodiments, in partial control of ventilation configuration, the ventilation system may sense a patient spontaneous breathing effort. According to some embodiments, in partial control of ventilation configuration, the ventilation system may apply only partial expansion force, which may compliment the patient's self-muscles force to yield collectively adequate chest wall dilation and inhalation.

According to some embodiments, in partial control of ventilation configuration, the ventilation system may sense a patient's self-spontaneous muscles work using various sensors, such as, for example, a pressure sensor, e.g., a piezoelectric sensor. According to some embodiments, the ventilation system may sense and/or measure the force (e.g., pressure) generated by the patient's muscles against the moveable unit of the system. According to some embodiments, the ventilation system may sense and/or measure the force (e.g., pressure) by an accelerometer sensor, which may be connected to a device arm and may sense the distance the arm has been moved by the patient's breathing muscles. According to some embodiments, such sensors and/or other sensors, e.g., stretch sensors, may be installed in an elastic belt which may be positioned around the patient's chest to measure (e.g., sense) chest dilation by surrogate and/or additional sensors. According to some embodiments, a carbon dioxide sensor, oxygen sensor, a sensor to detect nerve stimulation to the muscles. According to some embodiments, various sensors may be located at various positions in and/or on the patient's body and/or the device.

According to some embodiments, the information from the sensors may be collected in a data processing unit. According to some embodiments, the data processing unit may be part of the mechanical ventilation system. According to some embodiments, the data processing unit may be connected to the mechanical ventilation system remotely. According to some embodiments, the real-time sensing processing may be used for real-time automatic response by the ventilation system. According to some embodiments, the ventilation system may sense the initiation of spontaneous and/or ventilator-induced inspiration. According to some embodiments, the ventilation system may sense the rate of spontaneous breathing. According to some embodiments, the ventilation system may sense the magnitude of spontaneous patient breathing, in order to produce the best complementary mechanical responses, e.g., in terms of the rate, degree of expansion of the chest wall, and/or the overall behavior of the ventilation system during each, or at least one of the cycles of breathing.

According to some embodiments, in partial control of ventilation configuration, the ventilation system disclosed herein may be used for weaning from mechanical ventilation (for example by non-portable ventilators/breathing support devices). According to some embodiments, in the clinical setting, a non-portable mechanical ventilator may be used for the process of exchanging air between the lungs and the ambient air. According to some embodiments, a mechanical ventilator may function to exchange air between the lungs and the ambient air in patients faced with serious respiratory illness. According to some embodiments, once a patient is stable and/or in a condition to move towards spontaneous breathing it is important that steps be taken to wean the patient off dependency on breathing support devices. According to some embodiments, weaning may be the gradual withdrawal of a patient from assisted breathing on a life-support system and/or other form of respiratory treatment and/or therapy. According to some embodiments, weaning a patient from a ventilator may occur when the condition of the patient improves. According to some embodiments, a decision may be made to remove a patient from the ventilator through a trial of spontaneous breathing through the endotracheal tube and/or the tracheostomy tube and/or extubation (e.g., removal of the tube).

According to some embodiments, the goal during weaning may be to bring back the spontaneous physiologic breathing pacing and/or to bring breathing muscles back to full functioning. According to some embodiments, in partial control of ventilation configuration, the mechanical ventilation system may operate to support spontaneous breaths. According to some embodiments, support spontaneous breaths may be triggered by patient effort. According to some embodiments, once triggered, the ventilation system may provide the patient with only partial support, and not full support, i.e., may not provide full mechanical ventilation. According to some embodiments, an under-optimal mechanical and/or supportive ventilation may be provided to a patient during weaning, aiming to push the patient back to spontaneous breathing.

According to some embodiments, weaning may be a gradual reduction of mechanical ventilation assistance. According to some embodiments, weaning may relate to the termination of mechanical ventilation and/or to the removal of any artificial airway.

Advantageously, the ventilation system disclosed herein may be used as an optimal breathing supportive device which may be used for weaning mechanically ventilated patients due to its ability to accurately measure the spontaneous mechanical breathing work and/or may provide the optimal automatic supportive responses on each breathing cycle. Advantageously, the ventilation system may be set to provide different responses on each breathing cycle, including initiating the onset of inspiration time, and/or the involvement of the device during the inspiration. According to some embodiments, involvement of the mechanical ventilation system may be a dedicated support of the inspiration effort by gently expanding the sternum during the entire inspiration, and/or during part of the inspiration period. According to some embodiments, the patient's respiration gases may be monitored by a separate device and/or by the ventilation system (e.g., by the sensing and processing units thereof). According to some embodiments, the mechanical ventilation system may be equipped with a respirator measuring unit, which may be configured to measure Tidal Volume and/or gases (O₂ and CO₂) partial pressures in the inhaled and/or exhaled air.

According to some embodiments, the ventilation system may be implemented subcutaneously, intercostally, and/or within the thoracic cage.

Advantageously, the ventilation system may be used permanently and/or temporarily to support breathing. According to some embodiments, the mechanical ventilation system may be portable. According to some embodiments, the mechanical ventilation system may be implantable. According to some embodiments, the mechanical ventilation system may be partially implantable. According to some embodiments, the wearable suit may support and/or provide locate the drive device's base/s on a desired location of the body, for example on the clavicle and/or the pelvis (e.g., one or both sides of the pelvis). According to some embodiments, the wearable suit may be worn under and/or over a patient's clothes (e.g., as a vest). According to some embodiments, the patient may carry the mechanical ventilation system firmly attached to and/or partially or completely implanted within their body, e.g., to maintain breathing while the patient performs routine movements and in routine situations, such as, sitting, moving, walking, talking, etc.

According to some embodiments, there is provided a method for treating ventilation insufficiencies in a subject in need thereof, the method includes:
detecting initiation of spontaneous or ventilator-induced inspiration in the subject by at least one sensor;
processing data from the at least one sensor in real-time by a controller;
using the moveable unit in response to the processed data for applying a force expanding the chest cavity of the subject, thereby generating a negative pressure in a pleural space, leading to inspiration of air into a lung through an upper airway; and
releasing the applied force and allowing the chest cavity to return to its resting position, forcing the air out of the lung, thereby mimicking a spontaneous breathing cycle.

In some embodiments applying a force by the moveable unit includes retracting the subject's sternum in a vector that represents normal sternum movement during spontaneous breathing. In some embodiments retracting of the sternum facilitates contracting intercostal muscles of the chest cavity. Thus, the system disclosed herein can facilitate talking, coughing, coughing up sputum, or any other type of intentional air expulsion.

In some embodiments, the tidal volume of the breathing cycle is dependent on the force applied for expanding the chest cavity.

According to some embodiments, the method includes assisting the subject's own breathing efforts by applying the force for expanding the chest cavity. In some embodiments, the assisting may be applied at any time during inspiration. In some embodiments, the assisting may be applied on detecting the subject spontaneous breathing effort and applying partial force, thereby complimenting the subject's own breathing efforts to yield collectively adequate chest cavity expansion for inspiring air into the lung through the upper airways.

According to some embodiments, the method may further include gradually increasing the assistance applied on progression of a degenerative disease.

According to some embodiments, the method may further include weaning the subject from a standalone ventilator or ventilation-supportive device by gradually reducing the force applied for replacing the subject's own breathing efforts.

According to some embodiments, the detecting may include sensing the initiation of spontaneous or ventilator-induced inspiration, rate of spontaneous breathing, magnitude of spontaneous breathing, tidal volume of the breathing cycle, and any combination thereof. Each possibility is a separate embodiment.

According to some embodiments, the method may further include storing the data collected by the at least one sensor in the controller.

According to some embodiments, the method may further include monitoring the subject's own breathing efforts through the controller.

According to some embodiments, the method may further include adjusting the applied force in accordance with the subject's monitored own breathing efforts.

According to some embodiments, the method may further include controlling the applied force through the controller.

According to some embodiments, the method may further include performing the monitoring, adjusting and/or controlling remotely.

According to some embodiments, the method may further include performing the monitoring, adjusting and/or controlling by a health care provider.

According to some embodiments, there is provided a method for weaning a subject in need thereof from a standalone ventilator or ventilation-supportive device, the method includes:
detecting initiation of spontaneous or ventilator-induced inspiration in the subject using at least one sensor;
processing data from the at least one sensor in real-time by a controller;
using a moveable unit in response to the processed data for applying a force for assisting expansion of a chest cavity of the subject thereby generating a negative pressure in a pleural space, leading to inspiration of air into a lung through an upper airway;
releasing the force and allowing the chest cavity to return to its resting position, forcing the air out of the lung, thereby mimicking a spontaneous breathing cycle; and
gradually reducing the force applied for said assisting expansion of the chest cavity of the subject over a series of breathing cycles.

According to some embodiments, the weaning may include gradually strengthening intercostal muscles, retuning the intercostal muscles motor units, sending afferent triggering to the brain's breathing-related sensory, activating breathing-related centers, and any combination thereof.

According to some embodiments, the mechanical ventilation system may be used for weaning from mechanical ventilation. According to some embodiments, the mechanical ventilation system may be used as a supporting ventilation system, temporarily and/or permanently, in various degenerative pathologies, such as, Multiple Sclerosis, Amyotrophic Lateral Sclerosis, Spinal Muscular Atrophy, All Muscular Dystrophy (e.g., Duchenne Muscular Dystrophy, Becker Muscular Dystrophy etc.), Cervical Spine Injury, and the like.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

Unless otherwise defined, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein, the term “about” may be used to specify a value of a quantity or parameter (e.g., the length of an element) to within a continuous range of values in the neighborhood of (and including) a given (stated) value. According to some embodiments, “about” may specify the value of a parameter to be between 80% and 120% of the given value. According to some embodiments, “about” specifies the value of a parameter to be between 90% and 110% of the given value. According to some embodiments, “about” specifies the value of a parameter to be between 95% and 105% of the given value.

According to some embodiments, the controller unit includes a processing unit or module. According to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices. Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus. The processes and displays presented are not inherently related to any particular computer or other apparatus. Various general-purpose systems may be used with programs in accordance with the teachings herein, or it may prove convenient to construct a more specialized apparatus to perform the desired method(s). In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Or processing unit. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

In the description and claims of the application, each of the words “comprise” “include” and “have”, and forms thereof, are not necessarily limited to members in a list with which the words may be associated.

As used herein, the indefinite articles “a” and “an” mean “at least one” or “one or more” unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A mechanical system for improving, supporting and/or treating ventilation insufficiencies in a subject in need thereof, the system comprising:
a moveable unit configured to be attached/fixed to an anterior bony structure of a thoracic cavity, said moveable unit is physically and/or functionally associated with an actuator; and
a static component configured to anchor the actuator, and be connected to a posterior section of at least one rib;
wherein, when activated, the actuator is configured to displace the moveable unit and the attached bony structure, thereby affecting the volume of the thoracic cavity.

2. The system according to claim 1, wherein affecting the volume of the thoracic cavity comprises increasing the cavity volume, thereby forming negative pressure inducing inhalation into the lungs and/or decreasing the volume cavity, thereby inducing exhalation from the lung.

3. The system according to any one of claims 1-2, wherein the actuator comprises a stationary motor; and a moveable arm or sliding elements at least partially positioned on top of each other.

4. The system according to claim 3, wherein the arm or sliding elements are located posteriorly or inferiorly to the moveable unit.

5. The system according to any one of claims 1-4, wherein the actuator and the static component are positioned subcutaneously, externally to rib cage of the subject.

6. The system according to any one of claims 1-4, wherein the actuator and the static component are positioned within rib cage of the subject.

7. The system according to claim 3, wherein the motor comprises an electric motor, pneumatic piston or a hydraulic piston.

8. The system according to any one of claims 1-7, further comprising a diaphragm harness configured to prevent suction of the diaphragm into the thoracic cavity.

9. The system according to any one of claims 1-8, wherein the system further comprises a controller configured to activate the actuator.

10. The system according to any one of claims 1-9, wherein the system further comprises one or more biosensor(s) configured to detect initiation of spontaneous or ventilator-induced inspiration of the subject.

11. The system according to claim 10, wherein the one or more biosensor(s) are selected from: an oxygen sensor, a carbon dioxide sensor, a pressure sensor, a stretch sensor, an accelerometer, an electrical sensor to detect nerve and/or muscle stimulation or activity, Electromyography sensor (EMG), or any combination thereof.

12. The system according to any one of claim 10-11, wherein the one or more biosensors are positioned externally and/or internally in the subject's body.

13. The system according to any one of claims 1-12, further comprising a malfunction sensor, selected from, an accelerometer, a voltmeter, a current sensor, or any combinations thereof.

14. The system according to any one of claims 1-13, wherein the moveable unit comprises a fixation plate rigidly fixed to the anterior bony structure.

15. The system according to any one of claims 1-14, wherein the moveable unit is composed of a biocompatible material.

16. The system according to claim 15, wherein the biocompatible material is selected from: stainless steel, cobalt chrome alloys, titanium and titanium alloy, pyrolytic carbon, thermoplastics, bioceramics, and a combination thereof.

17. The system according to any one of claims 1-16, wherein the static component comprises at least one strap.

18. The system according to claim 17, wherein the at least one strap comprises two portions, each of the portions is configured to be anchored posteriorly at one end to at least one rib and anchored anteriorly at the other end to the actuator.

19. The system according to any one of claims 17-18, wherein the at least one strap is anchored posteriorly at one end to at least one rib by a plate and/or a hinge.

20. The system according to any one of claims 1-19, wherein the bony structure is an anterior rib or sternum of the subject.

21. The system according to any one of claims 1-20, wherein the moveable unit comprises a fixation plate configured to be attached to or fixed onto the bony structure of the thoracic cavity, wherein the relative vertical movement of the moveable unit, induced by the actuator, induces volume change of the thoracic cavity.

22. The system according to any one of claims 1-21, wherein at least part of the system is configured to be implanted subcutaneously.

23. The system according to any one of claims 1-22, wherein the controller is configured to activate the actuator by a predetermined amount, at predetermined timing and/or at a predetermined rate.

24. The system according to any one of claims 1-23, wherein the controller is configured to activate the actuator by an amount, timing and/or rate determined based on information from one or more sensors.

25. The system according to any one of claims 1-24, wherein the controller is configured to be worn externally by a user.

26. The system according to any one of claims 1-25, further comprising a rechargeable battery.

27. The system according to claim 26, wherein the rechargeable battery is configured to be worn externally by a user.

28. The system according to any one of claims 1-27, wherein the system is portable.

29. A method for supporting, improving and/or treating ventilation insufficiencies in a subject in need thereof, the method comprising:
implanting the ventilation system according to any one of claims 1-28 in the subject; and
using the controller to control operating parameters of the ventilation system.

30. The method according to claim 29, wherein the operating parameters comprise range of motion of the actuator, expansion volume of the thoracic cavity, timing of motion of the actuator, timing of expansion of the thoracic cavity, rate of motion of the actuator, rate of expansion of the thoracic cavity, or any combinations thereof.

31. The method according to claim 30, for supporting weaning of a subject from breathing support devices.

32. The method according to any one of claims 29-31, for improving self-breathing of the subject.

33. The method according to any one of claims 29-32, for facilitating talking and/or coughing of the subject.

34. The method according to any one of claims 29-33, wherein the subject is afflicted with a neurodegenerative condition.

35. A surgical method for implanting a system for improving, supporting and/or treating ventilation insufficiencies in a subject in need thereof, the system comprising a moveable unit configured to be attached/fixed to an anterior bony structure of a thoracic cavity, said moveable unit is physically and/or functionally associated with an actuator; and a static component configured to anchor the actuator, and be connected to a posterior section of at least one rib; the surgical method comprising the steps of:
fixing the moveable unit onto the anterior bony anterior bony structure of the thoracic cavity of the subject;
positioning the static component and the actuator;
associating the moveable unit and the actuator; and
controlling the operation of the actuator by an external controller, such that when activated, the actuator is configured to displace the moveable unit, thereby inducing movement of the bony structure and affecting the volume of the thoracic cavity;
to thereby at least partially control breathing of the subject.
